SEQUENCE LISTING (26 sequences)

SEQ ID NO: 1 - DNA, 37 bp, Artificial Sequence
  Synthetic
cgggatcccgtcatgattagtttaataaggaggatga

SEQ ID NO: 2 - DNA, 31 bp, Artificial Sequence
  Synthetic
cggcgccgcttcacggctgttaaattgatct

SEQ ID NO: 3 - DNA, 24 bp, Artificial Sequence
  Synthetic
ggaatggcgtgtgtgttagccaaa

SEQ ID NO: 4 - DNA, 24 bp, Artificial Sequence
  Synthetic
tcacacaggaaacagctatgacca

SEQ ID NO: 5 - DNA, 24 bp, Artificial Sequence
  Synthetic
ataggtggaaatgatgcgcttccg

SEQ ID NO: 6 - DNA, 22 bp, Artificial Sequence
  Synthetic
cccagcatatctgcaacttcct

SEQ ID NO: 7 - DNA, 24 bp, Artificial Sequence
  Synthetic
tcgcttcacgaatgcggttatctc

SEQ ID NO: 8 - DNA, 24 bp, Artificial Sequence
  Synthetic
ccaacttaatcgccttcgagcaca

SEQ ID NO: 9 - DNA, 56 bp, Artificial Sequence
  Synthetic
ccggatccacttgaatttaaaaggagggaacttagatggtatttgaaaaaattgat

SEQ ID NO: 10 - DNA, 28 bp, Artificial Sequence
  Synthetic
cggcgccagttacagacaaacctgaagt

SEQ ID NO: 11 - DNA, 24 bp, Artificial Sequence
  Synthetic
tggaaaggcaggtaaccttgaagc

SEQ ID NO: 12 - DNA, 24 bp, Artificial Sequence
  Synthetic
ctggcagttgtgtttccattcctc

SEQ ID NO: 13 - DNA, 24 bp, Artificial Sequence
  Synthetic
tggcgccacttaatgatttgccag

SEQ ID NO: 14 - DNA, 24 bp, Artificial Sequence
  Synthetic
tatctgacgtcaatgccgagcgaa

SEQ ID NO: 15 - DNA, 24 bp, Artificial Sequence
  Synthetic
tggaaaggcaggtaaccttgaagc

SEQ ID NO: 16 - DNA, 24 bp, Artificial Sequence
  Synthetic
agcagcttgtttccatcccagtct

SEQ ID NO: 17 - DNA, 24 bp, Artificial Sequence
  Synthetic
taaatgctacccttcggctcgctt

SEQ ID NO: 18 - DNA, 24 bp, Artificial Sequence
  Synthetic
atcttcgagggtcattccgcgatt

SEQ ID NO: 19 - DNA, 24 bp, Artificial Sequence
  Synthetic
gccgaaacattcggtttcatccca

SEQ ID NO: 20 - DNA, 24 bp, Artificial Sequence
  Synthetic
tggtttgtttgccggatcaagagc

SEQ ID NO: 21 - DNA, 24 bp, Artificial Sequence
  Synthetic
gctcttgatccggcaaacaaacca

SEQ ID NO: 22 - DNA, 24 bp, Artificial Sequence
  Synthetic
ctggcagttgtgtttccattcctc

SEQ ID NO: 23 - DNA, 82 bp, Artificial Sequence
  Synthetic
gatcctgtattaacgccgcaaatttcaaccttgtttataaccactttatcagcccctttgaaattaaaagtaattacattag

SEQ ID NO: 24 - DNA, 82 bp, Artificial Sequence
  Synthetic
gcgcctaatgtaattacttttaatttcaaaggggctgataaagtggttataaacaaggttgaaatttgcggcgttaatacag

SEQ ID NO: 25 - DNA, 30 bp, Artificial Sequence
  Synthetic
gtggttataaacaaggttgaaatttgcggc

SEQ ID NO: 26 - DNA, 26 bp, Artificial Sequence
  Synthetic
ccctataatcatcggaaccgcataag

(19) United States
(12) Patent Application Publication
(10) Pub. No.: US 2014/0335623 A1
(43) Pub. Date: Nov. 13, 2014

(54) METHODS AND COMPOSITIONS FOR GENERATING SPORULATION DEFICIENT BACTERIA

(71) Applicant: NORTHWESTERN UNIVERSITY, Evanston, IL (US)

(72) Inventors: Bryan P. Tracy, Newark, DE (US); Carlos J. Paredes, Sunnyvale, CA (US); Eleftherios T. Papoutsakis, Newark, DE (US)

(73) Assignee: NORTHWESTERN UNIVERSITY, Evanston, IL (US)

(21) Appl. No.: 14/274,252

(22) Filed: May 9, 2014

Related U.S. Application Data
(63) Continuation of application No. 12/485,636, filed on Jun. 16, 2009, now abandoned.
(60) Provisional application No. 61/061,845, filed on Jun. 16, 2008.

(51) Int. Cl. C12N 15/74

(52) U.S. Cl. CPC C12N 15/74
USPC 435/476; 435/252.7; 435/252.3

(57) ABSTRACT

The present invention relates to methods and compositions for engineering sporulating bacterial cells, particularly a cell of the class Clostridia. In particular, the present invention relates to the generation of sporulation deficient bacteria for the generation of industrial superior phenotypes.

### CROSS REFERENCE TO RELATED APPLICATIONS

This application is a continuation of U.S. application Ser. No. 12/485,636, filed Jun. 16, 2009, which claims priority to U.S. Provisional Application No. 61/061,845, filed Jun. 16, 2008, each of which are herein incorporated by reference in its entirety.

### GOVERNMENT SUPPORT

This invention was made with government support under BES-0418157 awarded by the National Science Foundation. The government has certain rights in the invention.

### FIELD OF INVENTION

The present invention relates to methods and compositions for engineering sporulating bacterial cells, particularly a cell of the class Clostridia. In particular, the present invention relates to the generation of sporulation deficient bacteria for the generation of industrial superior phenotypes.

### BACKGROUND OF THE INVENTION

The engineering of microbes for specialty chemical conversion, biofuel generation, bioremediation and pharmaceutical production remains an immediate scientific and industrial goal. Specifically for the class Clostridia among prokaryotes, the pursuit of industrial scale biofuel generation and Clostridia-based cancer therapies is motivating a tremendous amount of strain development. Clostridia are naturally some of the most prolific microorganisms for fermenting cellulosic material into valuable biofuel alcohols such as butanol and ethanol. Additionally, due to their anaerobic and spore forming characteristics, Clostridia are being engineered to target the necrotic and anaerobic cores of malignant tumors to kill tumors from the inside out.

The development of biofuel technologies has been on the scientific and technological agenda of our nation (and at the worldwide level) for over 35 years now (Ragauskas et al., Science, 2006. 311(5760): p. 484-9). The adoption of these technologies has been slow however, as they were more expensive than the use of finite, nonrenewable fossil fuels. Recently, several well-known geopolitical reasons and global warming concerns have shifted the focus of US energy consumption renewable sources.

Butanol is an important biofuel and biologically-produced chemical, driven by its superior chemical properties (e.g., compared to ethanol, it is less volatile and hydrophilic, more miscible with hydrocarbons, and of higher energy content per unit mass). Butanol can be biologically produced by the anaerobic ABE (Acetone-Butanol-Ethanol) clostridial fermentation (Jones and Woods, Microbiol Rev, 1986. 50(4): p. 484-524), which was a profitable industrial process until the early 1950s, when the petrochemical process took over. However, the announcement in June 2006 of the DuPont and British Petroleum (BP) joint venture for the industrial production and marketing of biobutanol as a biofuel marks the rebirth of the industrial ABE fermentation.

The traditional industrial process for butanol production was a batch fermentation (Jones and Woods, supra; Woods, Trends in Biotechnology, 1995. 13: p. 259-264; Durre, Appl Microbiol Biotechnol, 1998. 49: p. 639-648) in which butyric and acetic acids are produced first and once a critical concentration of undissociated butyric acid is achieved, acetone, butanol and ethanol are formed at the expense of the acetic and butyric acids. Unfortunately, low butanol titers, the relatively low selectivity (ratio of butanol to other solvents) for butanol, and the low productivity of batch bioreactors made this process economically unviable compared to the petrochemical method. Typical final butanol concentrations rarely exceeded 12-13 g/l (Marlatt and Datta, Biotechnology Progress, 1986. 2: p. 23-28) but economic analyses (Marlatt and Datta, supra; Lenz et al., Industrial & Engineering Chemistry Product Research and Development, 1980. 19(4): p. 478-483; Dadgar and Foutch, Biotechnology Progress, 1988. 4(1): p. 36-39) estimate that if final butanol concentrations of 19 g/l could be achieved it would cut the separation costs in half.

What is needed are improved strategies for engineering bacterial species for industrial biofuel production.

### SUMMARY OF THE INVENTION

The present invention relates to methods and compositions for engineering sporulating bacterial cells, particularly a cell of the class Clostridia. In particular, the present invention relates to the generation of sporulation deficient bacteria for the generation of industrial superior phenotypes.

For example, in some embodiments, the present invention provides a method for decoupling sporulation and solventogenesis in a sporulating bacterium (e.g., Clostridia such as C. acetobutylicum), comprising: contacting the bacterium with a vector (e.g., plasmid) comprising a nucleic acid that disrupts the function of at least one sporulation gene of the bacterium following homologous recombination. The present invention is not limited to a particular sporulation gene. Examples include, but are not limited to, sigma F (CAC2306), sigma E (CAC1695), sigma G (CAC1696), CAP0157, CAP0167, CAC3267, CAC1766, CAC2052, CAC0550, CAC2053 and CAP0166 and other sporulation genes (e.g., from other bacteria) or homologs of such genes, or processing proteins or any of the proteins required for its transcription and/or translation and/or obtaining a fully functional form of any of these genes. In some embodiments, knocks out the sporulation gene, mutates the sporulation gene or downregulates the expression of the sporulation gene following homologous recombination. Additionally examples of sporulation genes include, but are not limited to, all gene examples within related Clostridia species—C. beijerinckii NCIMB 8052 (GenBank # CP000721, Refseq NC_{—}009617); C. thermocellum ATCC27405 (GenBank # CP000568, Refseq NC_{—}009012); C. cellulolyticum H10 (GenBank # AAVC00000000, Refseq NZ_AAVC00000000, unfinished); C. butyricum 5521 (GenBank #ABDT00000000, Refseq NZ ABDT00000000, unfinished); C. phytofermentans ISDg (GenBank # CP000885, Refseq NC_{—}010001), In some embodiments, the nucleic acid integrates into the genome of the bacterium following homologous recombination. In some embodiments, the bacteria exhibits increased (e.g., at least 5%, 10%, 20%, 50%, 10%, 150%, 200%, 500%) solvent (e.g., butanol). production relative to the level of solvent production prior to the homologous recombination.

The present invention further provides a method for decoupling sporulation and solventogenesis in a sporulating bacterium (e.g., Clostridia such as C. acetobutylicum), comprising: contacting the bacterium with a nucleic acid that is at least partially complementary to at least one sporulation gene (e.g., those described herein) of the bacterium under conditions such that expression of the sporulation gene is reduced. In some embodiments, the nucleic acid is antisense RNA. In some embodiments, the bacteria exhibits increased solvent (e.g., butanol) production relative to the level of solvent production prior to the method.

Additional embodiments of the present invention provide a bacterial cell (e.g., Clostridia such as C. acetobutylicum), wherein the function of at least one sporulation gene (e.g., those described herein) of the bacterial cell is disrupted. In some embodiments, the sporulation gene is knocked out or mutated or the expression of the sporulation gene is down regulated. In some embodiments, the bacteria exhibits increased solvent (e.g., butanol) production relative to the level of solvent production of a wild type bacteria.

A method of producing a solvent, comprising culturing a bacterial cell (e.g., Clostridia such as C. acetobutylicum), wherein the function of at least one sporulation gene (e.g., those described herein) of the bacterial cell is disrupted, under conditions such that the bacterial cell produces solvent (e.g., butanol). Additional embodiments are described herein.

### DESCRIPTION OF THE FIGURES

FIG. 1. Transcriptional and putative activity profiles for the major sporulation factors. The standardized expression ratios compared to the RNA reference pool of sigH (A), spo0A (B), sigF (C), sigE (D) and sigG (E) and activity profiles based on the averaged standardized profiles of canonical genes under their control. Putative genes (based on the B. subtilis model) responsible for activating σ^{F} (spoIIE), σ^{E} (spoIIR), and σ^{G} (spoIIIA operon) (♦). (F) Compilation of the activity profiles for sigH, spo0A, sigF, sigE, and sigG. The numbers along the top correspond to the clusters in FIG. 1C, D and the bars indicate the timing of each cluster.

FIG. 2. Q-RT-PCR and microarray data comparison. RNA from a biological replicate bioreactor experiment was reverse transcribed into cDNA for the Q-PCR. All expression ratios are shown relative to the first timepoint for both Q-RT-PCR (◯) and microarray data (▪). Astericks represent data below the cutoff value for microarray analysis.

FIG. 3. Two possible scenarios for single crossover events. The above illustration shows what would occur if a single crossover occurred through the first or second region of homology. Additionally it illustrates what double crossover and plasmid excision events would result in.

FIG. 4. Expected product sizes from SigE integration confirmation primer set 1. Illustration of the expected product size when using primer set 1 in the SigE integration confirmation.

FIG. 5. Expected product sizes from SigE integration confirmation primer set 3. Illustration of the expected product size when using primer set 3 in the SigE integration confirmation.

FIG. 6. Expected product sizes from SigE integration confirmation primer set 4. Illustration of the expected product size when using primer set 4 in the SigE integration confirmation.

FIG. 7. PCR confirmation of SigE integration orientation. PCR results from the two SigE-KO mutants analyzed (8 and 15, mutant 3 was not obtained in this study), definitively conclude a single integration through the first region of homology because there is substantial product for primer sets 2 and 3.

FIG. 8. Microscopy of KOSIGE-2* and KOSIGE-2 cells during late stationary phase of growth. There are no visible phase bright morphologies in any field of view. Phase bright morphologies are indicative of developing endospores and mature spores. Moreover, there is obvious cell division occurring even as late as 122 hrs, indicative of metabolically active cells and continued solvent formation. The asterick denotes that the strain had undergone over 80 generations of vegetative growth prior to this culture, in order to simulate continuous culturing.

FIG. 9. Flow cytometry dot-plots of affected asRNA phenotypes. The dots represent the forward scatter and side scatter characteristics of all individual cells. Each plot consists of 10,000 such events.

FIG. 10. Microscopy results from asCAC1696-2 static flask. The majority of late-stage differentiating cells exhibit an elongated mother-cell compartment that is not characteristic of WT or pSOS95del cultures.

FIG. 11. Phase contrast microscopy of KOSIGG and WT cultures. A) Phase bright morphologies (dotted circle—A, early-stationary), similar to clostridial-cell forms and forespore-containing cells were witnessed during early stationary phase. At late-stationary phase, what appear as malformed (confirmed by TEM below) endospore-containing cells (dotted circle—A, late-stationary), were observed. B) Clostridial-cell forms (dotted circle—B, early-stationary) and well defined, phase bright, endospore-containing cells (dotted circle, B, late-stationary).

FIG. 12. Time course Flow-cytometry (FC) analysis of KOSIGG (denoted as ΔsigG in figure) and WT cultures. Gates: 1—low FSC (rod-shaped cells); 2—mid FSC (clostridial-cell forms & forespore-containing cells); 3—high FSC (endospore-containing cells and free spores) (Tracy et al., 2008). A) FC analysis of ΔsigG demonstrated that differentiation was stalled at endospore-containing cells. B) FC analysis of a typical WT time course.

### DEFINITIONS

To facilitate an understanding of the present invention, a number of terms and phrases are defined below:

As used herein, the term “increased solvent production” refers in an increase in solvent production by a solvent producing bacteria relative to a reference level such as the level of the wild type bacteria (e.g., before genetic manipulation of one or more sporulation genes). In some embodiments, solvent production is increased 5%, 10%, 20%, 50%, 10%, 150%, 200%, 500%, or more relative to the reference level.

As used herein, the terms “detect”, “detecting” or “detection” may describe either the general act of discovering or discerning or the specific observation of a detectably labeled composition.

As used herein, the term “gene transfer system” refers to any means of delivering a composition comprising a nucleic acid sequence to a cell or tissue. For example, gene transfer systems include, but are not limited to, vectors, microinjection of naked nucleic acid, polymer-based delivery systems (e.g., liposome-based and metallic particle-based systems) and the like.

As used herein, the term “site-specific recombination target sequences” refers to nucleic acid sequences that provide recognition sequences for recombination factors and the location where recombination takes place.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the terms “complementary” or “complementarity” are used in reference to polynucleotides (i.e., a sequence of nucleotides) related by the base-pairing rules. For example, the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second non-complementary target.

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

When used in reference to a single-stranded nucleic acid sequence, the term “substantially homologous” refers to any probe that can hybridize (i.e., it is the complement of) the single-stranded nucleic acid sequence under conditions of low stringency as described above.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tm of the formed hybrid, and the G:C ratio within the nucleic acids. A single molecule that contains pairing of complementary nucleic acids within its structure is said to be “self-hybridized.”

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under ‘medium stringency conditions,” a nucleic acid sequence of interest will hybridize only to its exact complement, sequences with single base mismatches, and closely relation sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

“High stringency conditions” when used in reference to nucleic acid hybridization comprise conditions equivalent to binding or hybridization at 42° C. in a solution consisting of 5×SSPE (43.8 g/l NaCl, 6.9 g/l NaH2PO4H2O and 1.85 g/l EDTA, pH adjusted to 7.4 with NaOH), 0.5% SDS, 5×Denhardt's reagent and 100 μg/ml denatured salmon sperm DNA followed by washing in a solution comprising 0.1×SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 g/l NaCl, 6.9 g/l NaH2PO4H2O and 1.85 g/l EDTA, pH adjusted to 7.4 with NaOH), 0.5% SDS, 5×Denhardt's reagent and 100 μg/ml denatured salmon sperm DNA followed by washing in a solution comprising 1.0×SSPE, 1.0% SDS at 42° C. when a probe of about 500 nucleotides in length is employed.

“Low stringency conditions” comprise conditions equivalent to binding or hybridization at 42° C. in a solution consisting of 5×SSPE (43.8 g/l NaCl, 6.9 g/l NaH2PO4H2O and 1.85 g/l EDTA, pH adjusted to 7.4 with NaOH), 0.1% SDS, 5×Denhardt's reagent [50×Denhardt's contains per 500 ml: 5 g Ficoll (Type 400, Pharamcia), 5 g BSA (Fraction V; Sigma)] and 100 μg/ml denatured salmon sperm DNA followed by washing in a solution comprising 5×SSPE, 0.1% SDS at 42° C. when a probe of about 500 nucleotides in length is employed.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.) (see definition above for “stringency”).

As used herein, the term “probe” refers to an oligonucleotide (i.e., a sequence of nucleotides), whether occurring naturally as in a purified restriction digest or produced synthetically, recombinantly or by PCR amplification, that is capable of hybridizing to at least a portion of another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

### DETAILED DESCRIPTION OF THE INVENTION

The present invention relates to methods and compositions for engineering sporulating bacterial cells, particularly a cell of the class Clostridia. In particular, the present invention relates to the genetic manipulation of sporulating bacteria for the generation of industrial superior phenotypes.

In some embodiments, the present invention provides Clostridium strains that enter the solvent production stage of cellular differentiation, but do not perform sporulation. For example, embodiments of the present invention reduce and/or eliminate the activity of sigE and sigG, directly. Both sigma factors are highly conserved amongst sporulating Gram-positive organisms (Nolling, et al., J Bacteriol, 2001. 183(16): p. 4823-38; Paredes et al., Nat Rev Microbiol, 2005. 3(12): p. 969-78; Sauer et al., FEMS Microbiol Rev, 1995. 17(3): p. 331-40; Sauer et al., J Bacteriol, 1994. 176(21): p. 6572-82; Wong et al. Gene, 1995. 153(1): p. 89-92), and their relevance in orchestrating transcriptional events related to sporulation in B. subtilis is well documented (Eichenberger et al., J Mol Biol, 2003. 327(5): p. 945-72; Steil et al., Microbiology, 2005. 151(Pt 2): p. 399-420; Stragier and Losick, Annu Rev Genet, 1996. 30: p. 297-41).

Recent studies have shown that differentiation programs of B. subtilis and C. acetobutylicum employ the same set of sigma factors for regulating differentiation (Sauer et al., 1994, supra; Sauer et al., 1995, supra; Wong et al., Gene, 1995. 153(1): p. 89-92; Tomas et al., Journal of Bacteriology, 2003. 185(15): p. 4539-4547; Santangelo et al., Fems Microbiology Letters, 1998. 161(1): p. 157-164). Moreover, their temporal sequence (Stragier and Losick, supra) seems to be conserved in solventogenic clostridia and in general in all solventogenic endospore formers. However many differences also exist (Paredes et al., supra; Wong et al., supra; Santangelo et al., supra), among others regarding the signals and mechanisms that trigger the expression of Spo0A seem to be different between both organisms (Paredes et al., supra; Alsaker and Papoutsakis, Journal of Bacteriology, 2005. 187(20): p. 7103-7118). In solventogenic clostridia, solvent formation requires the start of the sporulation program. This link occurs at the level of the master regulator of the sporulation cascade, i.e., the stage 0 sporulation protein A (Spo0A) and its disruption reduces acetone and butanol production to 2 and 8% of wild-type levels, respectively (Harris et al. Journal of Bacteriology, 2002. 184(13): p. 3586-3597).

An attempt for decoupling the two phenomena was reported whereby asRNA down-regulation was directed toward a SigE activating protein SpoIIE (Scotcher et al., J Bacteriol, 2005. 187(6): p. 1930-6; U.S. patent application Ser. No. 11/173,542). By downregulating SpoIIE, the investigators were able to delay sporulation and generate marginal improvements in butanol titers (Scotcher et al., supra), but they never abolished sporulation. Other attempts for generating a solvent producing, non-sporulating Clostridium strain are based on plasmid complementation of solvent producing genes in degenerate (non-sporulating and non-solvent forming) Clostridium strains (e.g. Nair and Papoutsakis, J Bacteriol, 1994. 176(18): p. 5843-6)). However, such approaches have not yet been able to generate strains that produce butanol at levels comparable let alone higher than the WT sporulating strains. Another approach is that of selection of natural mutants using continuous culture, which has been reported with a little characterized clostridium strain (ATCC 4259) (U.S. Pat. No. 4,521,516), which used to be called Clostridium acetobutylicum. Such an approach is based on unknown and likely unstable random mutations and is therefore of more limited value for long term applications, where further strain development could lead to substantial strain and process improvements. For example, this process cannot be applied to the Clostridium acetobutylicum ATCC 824 (type strain) because the genes for solvent production are located on the pSOL1 megaplasmid, which is typically lost upon extended continuous culture. Similarly the approach described in U.S. Pat. No. 5,191,673 uses chemical mutagenesis and results in an undefined mutant strain of the same strain ATCC 4259.

During experiments conducted during the course of development of embodiments of the present invention, by knocking out sigE (CAC1695) in Clostridium acetobutylicum ATCC824 (GenBank# AE001437 & AE001438, Refseq. NC_{—}003030 & NC_{—}001988), a non-sporulating strain with enhanced solvent production capabilities in comparison to the wild-type (WT) was generated. By down-regulating the transcription of sigG (CAC1696), via asRNA, a strain exhibiting less spore formation and enhanced solvent production in comparison to both WT and plasmid controls was generated. The sigG asRNA results indicate that by knocking out sigG sporulation is abolished and solvent formation is enhanced, as witnessed in the sigE knockout.

Embodiments of the present invention provide multiple, ideal, platform or endpoint strains for industrial scale continuous fermentation of low value biomass feedstocks into the alternative biofuel butanol. In addition, the compositions and methods of the present invention find use in the industrial production of, for example, butyric acid, butanediol, propanol, and acetoin by bacteria (e.g., clostridia).

### I. Alteration of Clostrida Sporulation Genes

As described above, embodiments of the present invention provide compositions and method for altering spore forming bacteria (e.g., Clostridia) for industrial scale biofuel production. In some embodiments, bacterial (e.g., Clostridia) strains are engineered that decouple solvent formation from sporulation. In some embodiments, such bacteria exhibit increased solvent production relative to a reference level (e.g., wild type level of the same bacteria prior to modification using the methods described herein). In some embodiments, solvent production is increased 5%, 10%, 20%, 50%, 10%, 150%, 200%, 500%, or more relative to the reference level.

In some embodiments, the function of one or more sigma factors, sigma factor processing proteins, or proteins required for the transcription and/or translation and/or obtaining a fully functional form of a sigma factor is disabled or eliminated in order to decouple solvent formation from sporulation. The present invention is not limited to a particular sporulation protein or sigma factor. Examples include, are not limited to, sigE (annotated as CAC1695 on C. acetobutylicum ATCC 824; GenBank #NC_{—}003030.1, GeneID 1117878), sigG (annotated as CAC1696 on C. acetobutylicum ATCC 824; GenBank #NC_{—}003030.1, GeneID 1117879), sigF (annotated as CAC2306 on C. acetobutylicum ATCC 824, GenBank #NC_{—}003030.1, GeneID 1118489), sigH, CAP0157 (GenBank AAK76902), CAP0167 (GenBank AAK76912), CAC3267 (GenBank #NC_{—}003030.1, GeneID 1119449), CAC1766 (GenBank #NC_{—}003030.1, GeneID 1117949, CAC2052 (GenBank #NC_{—}003030.1, GeneID 1118235), CAC0550 (GenBank #NC_{—}003030.1, GeneID 1116733), CAC2053 (GenBank #NC_{—}003030.1, GeneID 1118236), CAP0166 (GenBank AAK76911), C. beijerinckii NCIMB 8052 (GenBank # CP000721, Refseq NC_{—}009617); C. thermocellum ATCC27405 (GenBank # CP000568, Refseq NC_{—}009012); C. cellulolyticum H10 (GenBank # AAVC00000000, Refseq NZ_AAVC00000000, unfinished); C. butyricum 5521 (GenBank # ABDT00000000, Refseq NZ ABDT00000000, unfinished); C. phytofermentans ISDg (GenBank # CP000885, Refseq NC_{—}010001), other sporulation genes (e.g., from other bacteria) or homologs of such genes, or processing proteins or any of the proteins required for transcription and/or translation and/or obtaining a fully functional form of any of these genes.

Sigma factor function may be disrupted using any suitable method. In some embodiments, the gene encoding a sigma factor or sigma factor processing, transcription or translation factor gene is mutated or knocked out. Gene knock out or mutation may be accomplished using any suitable method, including but not limited to, homologous recombination.

Additional gene knock out techniques include, but are not limited to, the group II intron system referred to as ClosTron (Heap et al., Journal of Microbiological Methods, 2007. 70: p. 452-464; herein incorporated by reference in its entirety), multimeric, suicide plasmids (O'Brien and Melville, Infection and Immunity, 2004. 72(9): p. 5204-5215; herein incorporated by reference in its entirety) and monomeric suicide plasmids (Green et al. Microbiology, 1996. 142(pt. 8): p. 2079-2086; herein incorporated by reference in its entirety). RNA interference techniques include complementary RNA sequences (of variable length) that create double stranded RNA, which is either targeted for degradation or inhibits translation (Desai and Papoutsakis, Applied and Environmental Microbiology, 1999. 65(3): p 936-945; herein incorporated by reference in its entirety), and longer interference RNA that take in consideration terminal unpaired nucleotides, components, and loop degree of the resulting interference RNA (Tummala, Welker and Papoutsakis, Journal of Bacteriology, 2003. 185(6): p 1923-1934; herein incorporated by reference in its entirety).

### A. Homologous Recombination

In some embodiments, homologous recombination is used to disrupt the function of sporulation genes (e.g., sigma factor genes or related genes). Homologous recombination is routinely employed in molecular biology for a multitude of applications such as inserting recombinant genes into a host chromosome, targeting host genes for inactivation, and engineering host-reporter fusion proteins. More elegant genetic manipulation approaches employ homologous recombination to accelerate horizontal gene transfer (also known as lateral gene transfer) (Frost et al., Nat Rev Microbiol, 2005. 3(9): p. 722-32; Gogarten and Townsend, Nat Rev Microbiol, 2005. 3(9): p. 679-87; Smets and Barkay, Nat Rev Microbiol, 2005. 3(9): p. 675-8; Sorensen et al., Nat Rev Microbiol, 2005. 3(9): p. 700-10; Thomas and Nielsen, Nat Rev Microbiol, 2005. 3(9): p. 711-21).

Horizontal gene transfer refers to the phenomenon of genetic material transfer from one cell to another cell that is not its offspring. However, comparative genomics analyses indicate that Clostridia are a rare class of bacteria that do not contain genes for any recognizable resolvase protein (Rocha et al., PLoS Genet, 2005. 1(2): p. e15). In some embodiments, resolvase activity is re-introduced to Clostridia or other bacteria lacking resolvase systems via the recombinant expression of a resolvase protein (See e.g., U.S. application Ser. No. 12/437,985, Filed May 8, 2009, herein incorporated by reference).

In some embodiments, constructs are designed (See e.g., Example 2) that include the sequence for disrupting or knocking out the gene of interest as well as a resolvase gene to improve the efficiency of homologous recombination.

### B. Nucleic Acid Interference Techniques

In some embodiments, function of a gene involved in sporulation (e.g., sigma factors or genes involved in sigma factor processing) is disrupted using nucleic acid interference methods (e.g., antisense RNA, and related methods).

In other embodiments, expression of genes involved in sporulation is modulated using antisense compounds that specifically hybridize with one or more nucleic acids encoding the genes (See e.g., Georg Sczakiel, Frontiers in Bioscience 5, d194-201 Jan. 1, 2000; Yuen et al., Frontiers in Bioscience d588-593, Jun. 1, 2000; Antisense Therapeutics, Second Edition, Phillips, M. Ian, Humana Press, 2004; each of which is herein incorporated by reference).

### C. Kits

In some embodiments, the present invention provides kits for use in engineering bacteria such as Clostridia species to inhibit or eliminate expression of genes involved in sporulation. The kit may include any and all components necessary, useful or sufficient for engineering and screening bacteria including, but not limited to, the resolvase cassettes including sequences for targeted gene disruption, nucleic acid interference sequences, buffers, control reagents (e.g., bacterial samples, positive and negative control sample, etc.), reagents for screening for positive clones, labels, written and/or pictorial instructions and product information, inhibitors, labeling and/or detection reagents, package environmental controls (e.g., ice, desiccants, etc.), and the like. In some embodiments, the kits provide a sub-set of the required components, wherein it is expected that the user will supply the remaining components. In some embodiments, the kits comprise two or more separate containers wherein each container houses a subset of the components to be delivered.

### II. Uses

Embodiments of the present invention find use in a variety of applications for use of bacteria (e.g., Clostridia) for industrial production of biofuels and other chemicals. Examples include, but are not limited, to 1) fermentative production of chemical feedstocks for subsequent synthesis into acrylate/methacrylate esters, glycol ethers, butyl-acetate, amino resins and butylamines; 2) fermentative conversion of biodiesel glycerol waste streams to propionic acids; 3) fermentative production of acetone, ethanol and/or butanol production as bulk chemicals; 4) fermentative production of butanol and/or ethanol as a transportation fuel (biofuel); and 5) fermentative production of all aforementioned chemical species from renewable resources such as cellulosic and hemicellulosic materials.

Additional uses are within the scope of one of skill in the art.

### EXPERIMENTAL

The following examples are provided in order to demonstrate and further illustrate certain preferred embodiments and aspects of the present invention and are not to be construed as limiting the scope thereof.

### Example 1

### Transcriptional Profiling

To capture the transcriptional, physiological, and morphological changes (Alsaker and Papoutsakis, Journal of Bacteriology, 2005. 187(20): p. 7103-7118; Jones et al., Applied and Environmental Microbiology, 1982. 43(6): p. 1434-1439) occurring during the C. acetobutylicum sporulation process, RNA samples were taken every hour during exponential phase and every two hours after, until late stationary phase. A total of 25 timepoints were selected for transcriptional analysis by hybridizing pairs of 22 k oligonucleotide microarrays on a dye swap configuration using an mRNA pool as reference.

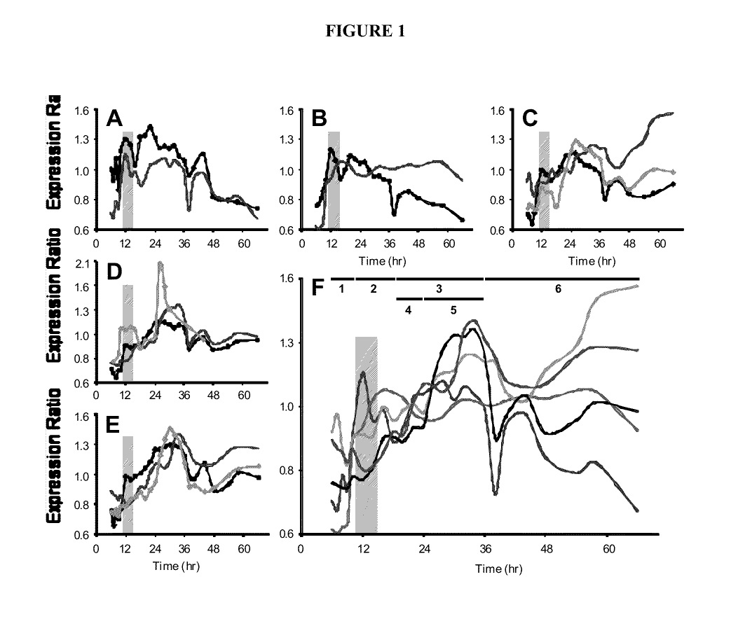

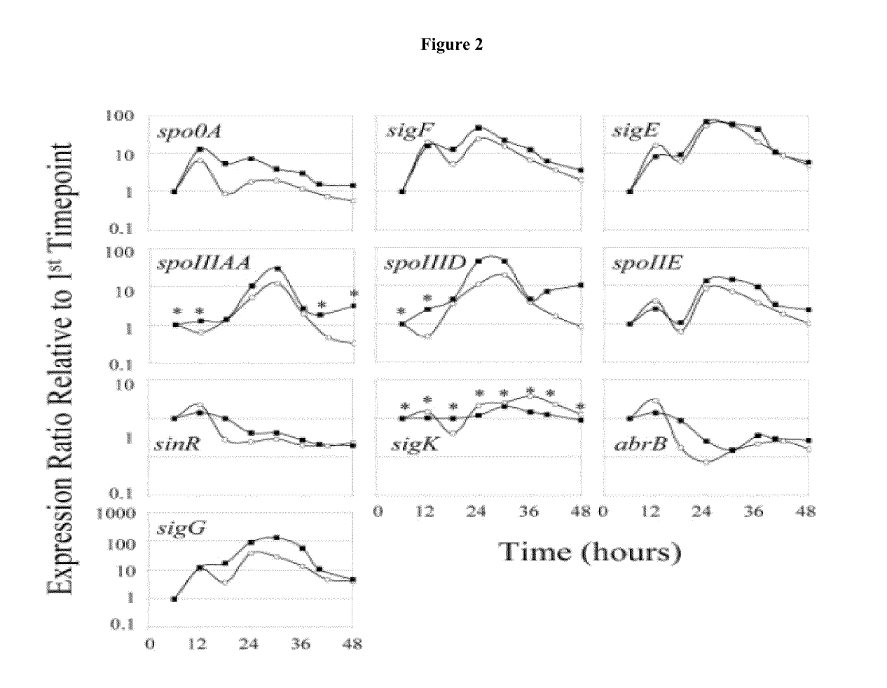

Bacilli sporulation is controlled by the conserved, master transcriptional regulator, Spo0A (Paredes et al., supra). spo0A expression peaked at hour 12 and maintained a minimum of 3-fold induction, relative to the first timepoint, until hour 36 (FIG. 1). Once phosphorylated in C. acetobutylicum, Spo0A regulates the expression of the operons encoding sigF, sigE, and spoIIE (Molle et al., Molecular Microbiology, 2003. 50(5): p. 1683-1701). sigF and sigE exhibit an initial induction at hour 12, the timing of peak spo0A expression, but a second higher level of induction is reached later at hour 24 (FIG. 1). This bimodal pattern was verified by Q-RT-PCR (FIG. 2). The second level of induction coincides with decrease in expression of two known repressors of sporulation genes, abrB and sinR, the latter of which, in B. subtilis, binds to the promoters of both sigF and sigE operons (Wong et al., supra; Dadgar et al., Biotechnology Progress, 1988. 4(1): p. 36-39). In B. subtilis, Spo0A either indirectly (sinR) or directly (abrB) represses the genes of these two repressors (Mandicmulec et al., Journal of Bacteriology, 1995. 177(16): p. 4619-4627; Cervin et al., Nucleic Acids Research, 1998. 26(16): p. 3806-3812), but the expression of spo0A (and the deduced Spo0A˜P activity, see below) is much earlier than the down-regulation of these genes (FIG. 1).

sigF, sigE and sigG have very similar expression patterns (FIG. 1). Similar expression profiles of sigF and sigE are expected based on their regulation by Spo0A, but in B. subtilis, both σF and σE are necessary for sigG transcription (Stragier and Losick, supra). However, sigE and sigG are computationally predicted to belong to a single operon in C. acetobutylicum (Paredes et al., Nucleic Acids Research, 2004. 32(6): p. 1973-1981 and Northern analysis confirms this (Harris et al., Journal of Bacteriology, 2002. 184(13): p. 3586-3597). All three sigma factor expression profiles were independently confirmed by Q-RT-PCR from a biological replicate experiment (FIG. 2).

### Example 2

### sigE Knockout in WT Background

Construction of sigE Targeted Gene Disruption Plasmid

For the C. acetobutylicum sigE gene (CAC1695) targeted plasmid, the disrupted sigE gene fragment was constructed in the pCR8-GW-TOPOTA™ cloning plasmid from Invitrogen. A 559 bp region of the sigE gene was PCR amplified with Taq polymerase and SigE-F/R primer set, and then cloned into the pCR8-GW-TOPOTA™ cloning plasmid and One Shot® TOP10 E. coli via manufacturer suggestions. The resulting plasmid is called pCR8-SigE. The sigE gene fragment was then disrupted in approximately the middle of the gene fragment via a NdeI endonuclease digestion. The linear plasmid was blunt ended via NEB® Klenow (large fragment) treatment and then dephosphorylated. An antibiotic cassette was cloned into the linear plasmid via NEB Quick Ligase and cloned into Invitrogen® One Shot® TOP10 E. coli. The antibiotic cassette for the sigE disruption was a modified chloramphenicol/thiamphenicol (CM/TH) marker described later. The resulting plasmid is designated pCR8-SigE/CM/ptB. The SigE/CM/ptB gene disruption cassette was PCR amplified out of pCR8-SigE/CM/ptB with the SigE-F/R primer set and Vent polymerase for blunt end product. The replicating plasmid backbone with the resolvase cassette was prepared by double digesting pRecU with AvaII and XcmI, and gel band purifying the resulting 4398 bp product. This plasmid backbone was blunt ended via NEB® Klenow (large fragment) treatment and then dephosphorylated. The 1610 bp SigE/CM/ptB gene disruption cassette was ligated into the pRecU backbone via NEB Quick Ligase and cloned into Invitrogen® One Shot® TOP10 E. coli. The final replicating, sigE targeted plasmid is called pKORSIGE.

### Construction of Resolvase Cassette

The resolvase cassette was constructed by cloning the recU (BSU22310) open reading frame (ORF) plus native Shine-Dalgarno (SDG) sequence from B. subtilis ATCC23857 (GenBank # AL009126, Refseq NC_{—}000964) into pSOS95del via a directional sticky end ligation of BamHI and KasI. The recU and engineered BamHI and KasI digest sites were amplified from B. subtilis ATCC23857 genomic DNA with the recU-F and recU-R primer set. The 719 bp PCR product was purified, double digested with BamHI and KasI, and phosphorylated. pSOS95del was generating by double digesting pSOS95 with BamHI and KasI, gel band purifying the 4979 bp plasmid backbone, and dephosphorylating. The pSOS95del plasmid backbone and recU PCR product were ligated via New BioLabs® (NEB) Quick Ligase and cloned into Invitrogen® One Shot® TOP10 E. coli. The resulting plasmid we call pRecU. The resolvase cassette was PCR amplified out of pRecU with the recU-cass-F and recU-cass-R primer set and NEB Vent polymerase for blunt end product.

### Construction of the Modified TH Marker

A new CM/TH antibiotic marker was constructed, which replaced the old SDG with an optimal SDG and placed its expression under the transcriptional control of either the thL or phosphotransbutyrylase promoter (ptB). A 1567 bp region was PCR amplified from pLHKO [34] with CM-F and CM-R primers. This region contains the annotated CM/TH marker, including the associated promoter and terminator regions. This serves as the unmodified antibiotic marker. A 687 bp modified CM/TH marker was generated from the 1567 bp region by PCR with mod-CM/SDG-F and mod-CM/SDG-R primers. The CM/TH modified marker includes the following: the 624 bp ORF, a newly designed Shine-Delgarno sequence (SDG), a 5′-BamHI restriction site and a 3′-KasI restriction site. The mod-CM/SDG-F primer included 33 bps of homology to the original CM/TH marker, including the ATG start codon, 6 additional codons, and 12 bps upstream of the start codon. It also included 23 bps of new sequence on the 5′-end of the primer that coded for a new “more conserved” SDG and a BamHI restriction site. The mod-CM/SDG-R primer consisted of 21 bps of homology to the CM/TH marker, specifically the last by of the ORF and 20 additional non-coding bps of homology, and 7 new nucleotides on the primer 5′-end encoding a KasI restriction site. Resulting PCR product was double digested with BamHI and KasI and directionally cloned into either pSOS94del or pSOS95del, for ptB or thL promotion respectively. pSOS95del was generated as described in “Construction of resolvase cassette,” and the pSOS94del is the exact same plasmid backbone but with the ptB promoter instead of thL. The modified antibiotic cassettes were then PCR amplified out of the resulting p95CM and p94CM plasmids with the recU-F/R primer set.

Generation of sigE Disruption Mutants

Targeted gene disruption plasmid was transformed into C. acetobutylicum via a previously reported electroporation protocol (Mermelstein et al., Bio-Technology, 1992. 10(2): p. 190-195). Prior to transforming, plasmid DNA was site specifically methylated to avoid degradation by the clostridial endonuclease CAC8241. Plasmid DNA was methylated by shuttling through E. coli ER2275 pAN2. pAN2 contains a gene encoding for the site-specific methyltransferase.

Transformants were vegetatively transferred every 24 hrs for 5 days via replica plating on solid 2xYTG plates supplemented with the antibiotic disrupting the gene of interest. For pKORSIGE a thiamphenicol (TH_antibiotic marker is disrupting the gene fragment and an erythromycin (EM) marker is on the backbone of the plasmid. So, vegetative transfers were performed under TH selection. Antibiotic concentrations were 40 μg/mL for EM and 20 μg/mL for TH. After five days, the cells were again vegetatively transferred for an additional five days under no antibiotic selection. This is performed for plasmid curing (to lose the plasmid). After five days of curing, the cells were transferred to plates containing the antibiotic disrupting the gene of interest, and allowed to grow for 24 hrs. These plates were then transferred to plates supplemented with the antibiotic on the vector backbone, allowed to grow for 24 hrs and compared to the previous plates. Areas of growth and no growth on the plates supplemented with the antibiotic disrupting the gene of interest and antibiotic on the vector backbone, respectively, were indicative of chromosomal integrations and more specifically double crossover events. These putative gene disruptions were streaked on plates supplemented with the antibiotic disrupting the gene of interest, allowed to grow for 24 hrs, and then replica plated onto the other antibiotic plate in order to clearly demonstrate antibiotic sensitivity.

### Confirming Gene Disruption Mutants

Gene disruption mutants were confirmed by PCR amplification of the region in which the plasmid integrated and then DNA sequencing. Sequencing primers are given in Table 5. Plasmids and primers used in the described experiments are shown in Table 6.

TABLE 5
Primer and asRNA oligo sequences employed in this study

| | | | SEQ |
|---|---|---|---|
| | | | ID |
| Sequence Name | Sequence (5′-3′) | Description | NO |
| recU-F | CGGGATCCCGTCATGATTAGTTTA | FP to amplify the recU gene (BSU22310) | 1 |
| | ATAAGGAGGATGA | from B. subtilis ATCC23857 genomic DNA | |
| | | (GenBank# AL009126; Refseq NC_000964) | |
| | | and a BamHI endonuclease recognition site | |
| recU-R | CGGCGCCGCTTCACGGCTGTTAAA | RP to amplify the recU gene (BSU22310) | 2 |
| | TTGATCT | from B. subtilis ATCC23857 genomic DNA | |
| | | (GenBank# AL009126; Refseq NC_000964) | |
| | | and a KasI endonuclease recognition site | |
| recU-cass-F | GGAATGGCGTGTGTGTTAGCCAAA | FP to amplify recU out of pSOS94del or | 3 |
| | | pSOS95del | |
| recU-cass-R | TCACACAGGAAACAGCTATGACCA | RP to amplify recU out of pSOS94del or | 4 |
| | | pSOS95del | |
| SigE-F | ATAGGTGGAAATGATGCGCTTCCG | FP to amplify a portion of CAC1695 from | 5 |
| | | C. acetobutylicum ATCC824 genomic DNA | |
| | | GenBank# AE001437; Refseq NC_003030) | |
| SigE-R | CCCAGCATATCTGCAACTTCCT | RP to amplify a portion of CAC1695 from | 6 |
| | | C. acetobutylicum ATCC824 genomic DNA | |
| | | (GenBank# AE001437; Refseq NC_003030) | |
| CM-F | TCGCTTCACGAATGCGGTTATCTC | FP to amplify 1567bp | 7 |
| | | Chloramphenicol/Thiamphenicol antibiotic | |
| | | gene | |
| CM-R | CCAACTTAATCGCCTTCGAGCACA | RP to amplify 1567bp | 8 |
| | | Chloramphenicol/Thiamphenicol antibiotic | |
| | | gene | |
| mod-CM/SDG-F | CCGGATCCACTTGAATTTAAAAGG | FP to amplify 687bp novel | 9 |
| | AGGGAACTTAGATGGTATTTGAAA | Chloramphenicol/Thiamphenicol antibiotic | |
| | AAATTGAT | gene | |
| mod-CM/SDG-R | CGGCGCCAGTTACAGACAAACCTG | RP to amplify 687bp novel | 10 |
| | AAGT | Chloramphenicol/Thiamphenicol antibiotic | |
| | | gene | |
| SigE-KO-conf-F | TGGAAAGGCAGGTAACCTTGAAGC | FP to confirm SigE gene disruption | 11 |
| SigE-KO-conf-R | CTGGCAGTTGTGTTTCCATTCCTC | RP to confirm SigE gene disruption | 12 |
| SigE-Seq-PS1-F | TGGCGCCACTTAATGATTTGCCAG | SigE integration sequencing PS1 F | 13 |
| SigE-Seq-PS1-R | TATCTGACGTCAATGCCGAGCGAA | SigE integration sequencing PS1 R | 14 |
| SigE-Seq-PS2-F | TGGAAAGGCAGGTAACCTTGAAGC | SigE integration sequencing PS2 F | 15 |
| SigE-Seq-PS2-R | AGCAGCTTGTTTCCATCCCAGTCT | SigE integration sequencing PS2 R | 16 |
| SigE-Seq-PS3-F | TAAATGCTACCCTTCGGCTCGCTT | SigE integration sequencing PS3 F | 17 |
| SigE-Seq-PS3-R | ATCTTCGAGGGTCATTCCGCGATT | SigE integration sequencing PS3 R | 18 |
| SigE-Seq-PS4-F | GCCGAAACATTCGGTTTCATCCCA | SigE integration sequencing PS4 F | 19 |
| SigE-Seq-PS4-R | TGGTTTGTTTGCCGGATCAAGAGC | SigE integration sequencing PS4 R | 20 |
| SigE-Seq-PS5-F | GCTCTTGATCCGGCAAACAAACCA | SigE integration sequencing PS5 F | 21 |
| SigE-Seq-PS5-R | CTGGCAGTTGTGTTTCCATTCCTC | SigE integration sequencing PS5 R | 22 |
| CAC1696-asRNA-S | GATCCTGTATTAACGCCGCAAATT | SigG asRNA sense strand | 23 |
| | TCAACCTTGTTTATAACCACTTTA | | |
| | TCAGCCCCTTTGAAATTAAAAGTA | | |
| | ATTACATTAG | | |
| CAC1696-asRNA-AS | GCGCCTAATGTAATTACTTTTAAT | SigG asRNA antisense strand | 24 |
| | TTCAAAGGGGCTGATAAAGTGGTT | | |
| | ATAAACAAGGTTGAAATTTGCGGC | | |
| | GTTAATACAG | | |

TABLE 6
Table of strains and plasmids employed in this study.

| Strain or Plasmid Name | Relevant Characteristics | Source |
|---|---|---|
| Strain | | |
| E. coli One Shot Chemically | Invitrogen competent cells | Invitrogen |
| Competent TOP10 | | |
| E. coli ER2275 | recA lacZ mcrBC | NEB |
| ATCC824 | type strain | ATCC |
| KOSIGE | ATCC824 sigE::Th^{r} | this study |
| Plasmids | | |
| pSOS95 | Amp^{r} MLS^{r}; repL ori; ace2 operon under thL promoter | Tummala et al. 1999 |
| pSOS95del | Amp^{r} MLS^{r}; repL ori; thL promoter | Tummala et al. 2003 |
| pSOS94 | Amp^{r} MLS^{r}; repL ori; ace2 operon under ptB promoter | Tummala et al. 1999 |
| pSOS94del | Amp^{r} MLS^{r}; repL ori; ptB promoter | Tummala et al. 1999 |
| pRecU | Amp^{r} MLS^{r}; repL ori; recU under thL promoter | this study |
| pAN2 | Amp^{r}; carries the φ3TI gene | Tomas, C. (unpublished) |
| pCR8-GW-TOPOTA | Sp^{r}; topoisomerized; ori | Invitrogen |
| pCR8-SigE | pCR8-GW-TOPOTA with sigE fragmentcloned | this study |
| pCR8-GW-SigE/CM/ptB | pCR8-GW-TOPOTA with sigE::modified Th^{r} | this study |
| pKORSIGE | Amp^{r} MLS^{r}; repL ori; recU under thL promoter; sigE::Th^{r} | this study |
| pLHKO | Th^{r}; repL ori | Harris et al. |
| p95CM | Amp^{r} MLS^{r}; repL ori; Cm/Th^{r} under thL promoter | this study |
| p94CM | Amp^{r} MLS^{r}; repL ori; Cm/Th^{r} under ptB promoter | this study |
| pAS-CAC1696 | Amp^{r} MLS^{r}; repL ori; CAC1696 asRNA operon under thl promoter | this study |

ace2 operon, synthetic operon which contains the three acetone formation genes (adc, ctfA, and ctfB) transcribed from the adc promoter from ATCC824 (AE001437);
Amp^{r}, ampicilin resistance gene;
DEST cassette, Invitrogen Destination cassette for Gateway ™ cloning system;
MLS^{r}, macrolide-lincosamide-streptogramin resistance gene;
repL, pIM13 gram-positive origin of replication;
ori, ColE1 origin of replication;
recU, resolvase ORF and Shine-Delgarno sequence (BSU22310) from B. subtilis ATCC23857 (GenBank# AL009126; Refseq NC_000964);
Sp^{r}, spectinomycin resistance gene;
Th^{r}, thiamphenicol and chloramphenicol resistance gene;
φ3TI, Bacillus subtilis phage φ3TI methyltransferase gene
NEB, New England Biolabs, Beverly, MA.
ATCC, American Type Culture Collection, Manassas, VA.

Results from sigE Disruption Mutants

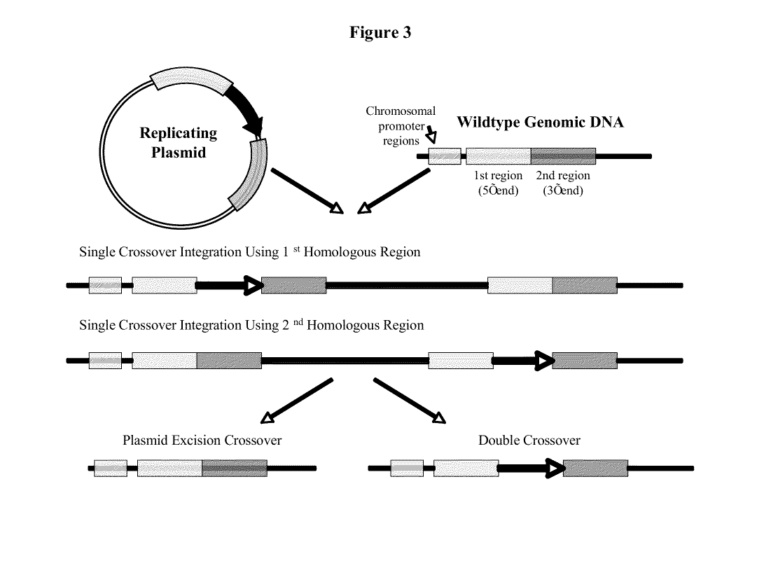

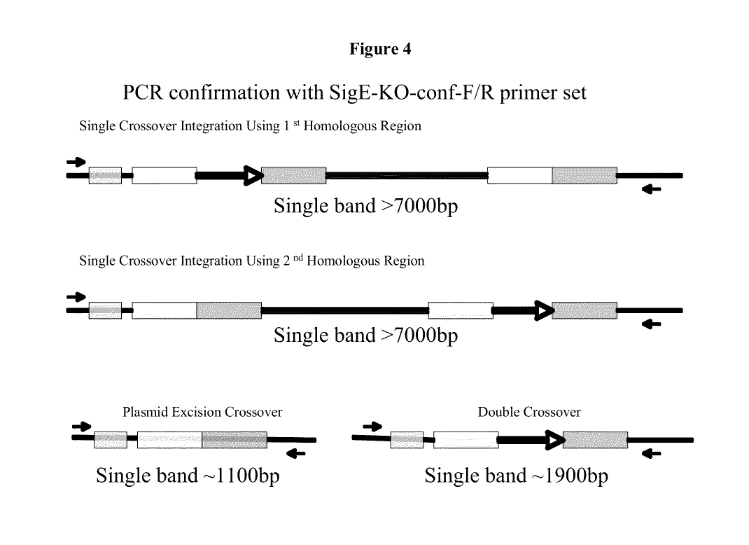

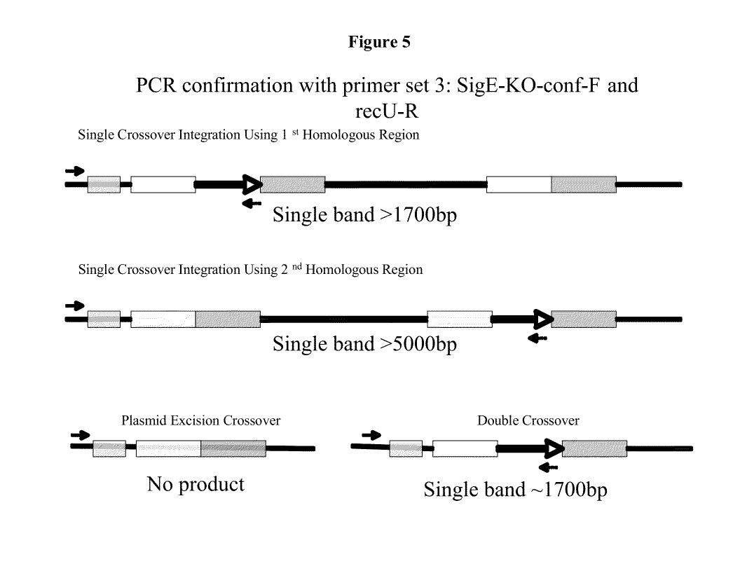

Numerous putative gene disruption mutants resolved on the final TH plating following the complete replica plating protocol. These mutants were identified by comparing to the EM plate after 24 hrs of growth. However, the majority of these regions on the EM plate actually showed growth after 72 hrs of incubation. The explanation is that a single crossover gene disruption event took place. In the case of a single crossover event, the entire plasmid gets incorporated into the chromosome and its orientation is dependent on which region of homology underwent crossover. Therefore both antibiotic markers were incorporated into the chromosome. However, since the EM marker was not under the control of a strong Clostridia promoter and present as only a single copy (plasmid was lost by this time), it took longer than 24 hrs for strains harboring a single chromosomal copy of the EM gene to grow on EM plates. PCR confirmation of gene disruption was performed for two of these mutants. Results indicated that the first region of homology (5′-end of the sigE gene) had performed the crossover, which effectively disrupted any full copy of the sigE gene. If a single crossover occurred and the entire plasmid incorporated, the confirmation PCR would result in a PCR product ˜7000 bp large. Obtaining PCR product of this size is difficult, thus primer sets that could only amplify PCR product if the plasmid had incorporated into the chromosome at the desired location were used. Specifically, the following primer sets were used: 1) SigE-KO-conf-F and SigE-KO-conf-R; 2) recU-F and recU-R; 3) SigE-KO-conf-F and recU-R; and 4) SigE-KO-conf-R and recU-F. Refer to FIGS. 3-7 for a schematic explanation and PCR results.

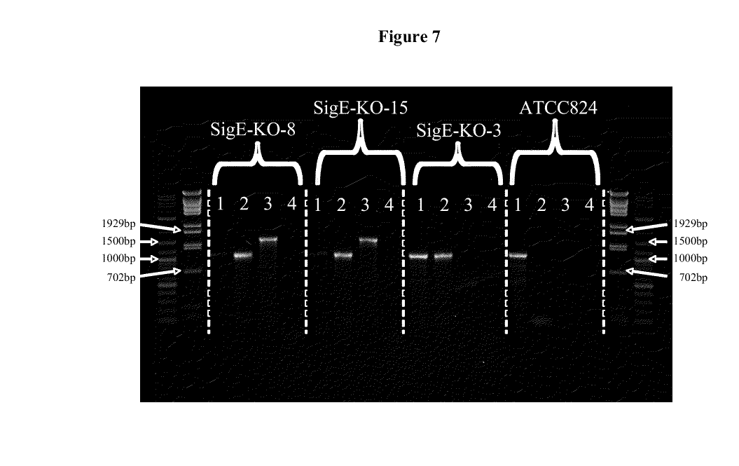

In the case of no integration, one should witness an intense ˜1000 bp PCR product band for primer set 1 when running PCR product on an agarose gel. There should not be any product band for any other primer set. This was the case for the WT genomic DNA template. If any sort of incorporation has occurred in the genome, one should be able to readily amplify out the TH marker with primer set 2 and resolve an intense ˜1000 bp PCR product. This is what was witnessed from both mutant DNA templates, and there is no product for WT template, as expected. If integration occurred through the first region of homology, a ˜1700 bp product should be amplified with primer set 3. This PCR product includes the 5′-flanking region of the chromosome, the first region of homology and the entire TH marker. If integration occurred through the first region of homology one could also theoretically amplify a >5000 bp region with primer set 4. This PCR product consists of the 3′-flanking region of the chromosome, the entire 3′-coding region of the gene up to the point where the first region of homology incorporated, the vector backbone, the second region of homology and the TH marker. If integration occurred through the second region of homology, one should readily amplify a ˜1700 bp product with primer set 4. This PCR product consists of the 3′-flanking region of the chromosome, the second region of homology and the TH marker. A >5000 bp region can also be amplified with primer set 3. This PCR product consist of the 5′-flanking region of the chromosome, the coding region of the gene to where the second region of homology ends, the vector backbone, the first region of homology and the TH marker. The >5000 bp products are not going to amplify because the small PCR product will out compete the large PCR production for dNTPs. Thus, if integration has occurred, one would expect no product band primer set 1, an intense product band for primer set 2 and a single intense product band for either primer set 3 or 4 but not both. For both mutant DNA templates the results indicate single integration through the first region of homology, refer to FIG. 7.

In order to definitively confirm, regions about the chromosome that extend into the plasmid integrated DNA were PCR amplified and sent for sequencing. Sequencing primer sets are provided in Table 5. Sequencing results conclusively proved a single integration through the first region of homology.

Morphology Results from sigE Single Crossover Disruption

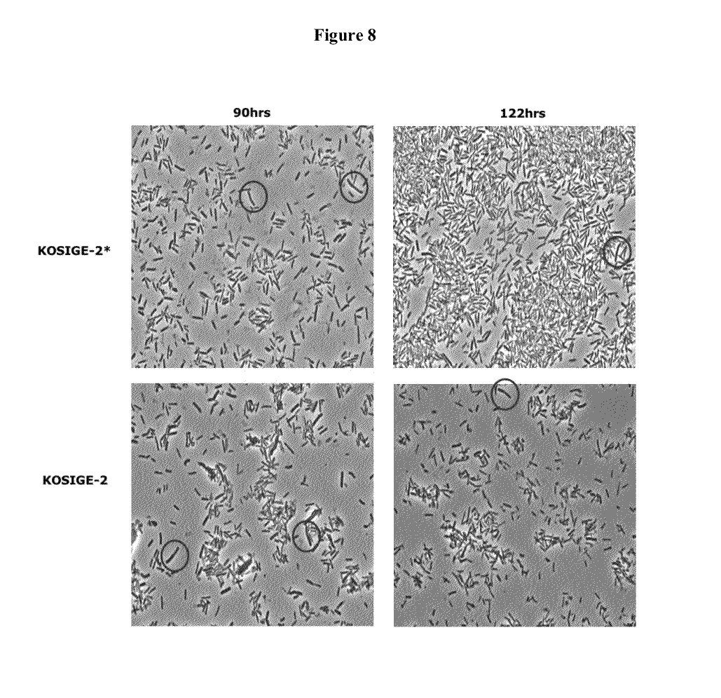

sigE mutants are referred to as KOSIGE. The resulting morphology was examined via phase contrast microscopy to the known sigE deletion mutant phenotype of the best-studied relative, B. subtilis. There exist readily identified homologs to all the important sporulation associated sigma factors from B. subtilis in C. acetobutylcum (Paredes et al., supra). In the case of a sigE disruption in B. subtilis, cells are arrested at the forespore stage of sporulation (Stragier and Losick, supra). An asporogenous phenotype was confirmed via phase contrast microscopy and flow cytometry, refer to FIG. 8. There was absolutely no spore formation throughout the entire life of the culture, when carried out for 170 hrs. Moreover, cells appeared to be undergoing normal vegetative cellular division at 122 hrs, which is very unusual and an obvious sign of continued metabolism.

### KOSIGE HPLC Analysis—Proving Enhanced Solvent Formation

After isolating KOSIGE, multiple tube and static flask cultures were performed to characterize the resulting solvent formation phenotype. The initial, individual tube culture generated 194 mM butanol, compared to 166 mM and 151 mM for WT and plasmid control tube cultures, respectively (refer to Table 1). This translates into a >116% and >128% benefit in butanol production compared to WT and plasmid control, respectively. Moreover, remaining glucose was <20 mM, compared to 100-130 mM for typical WT and pSOS95del cultures. The low amount of remaining glucose indicates that the culture remains metabolically active much longer than WT and plasmid control cultures.

Four biological replicate static flasks were performed. Two replicates were from KOSIGE frozen stocks and two others were from cultures that had previously been vegetatively transferred over 80 generations without antibiotic for testing pseudo-continuous cell culture stability and integration stability (these are referred to as KOSIGE-1* or KOSIGE-2*). Flow-cytometry and microscopy analysis again confirmed that absolutely no late-stage differentiation morphologies evolved, refer to FIG. 8. Additionally, there was obvious cell division occurring as late as 122 hrs. Analysis of the final butanol and glucose concentrations clearly show a large amount of solvent production, which was significantly higher than the WT average for both the cultures started from frozen stocks and cultures that had previously been vegetatively transferred over 80 generations, refer to Table 2.

TABLE 1
Final butanol concentrations compared to KOSIGE. The final butanol
concentration for KOSIGE comes from a single tube culture,
but the WT and pSOS95del control concentrations are from
an average of at least 3 cultures.
Average Final Butanol Concentration

| Strain | Mmol BuOH Final |
|---|---|
| pSOS95 del control | 151 |
| ATCC824 (WT) | 166 |
| KOSIGE | 194 |

TABLE 2
Final butanol and glucose concentrations for KOSIGE biological
replicates (half after being transferred without antibiotic selection for
80 generations to examine stability of the recombinant strains)
compared to WT. ATCC824 culture average was calculated from
data from two static flask experiments.

| | | Butanol | | Glucose |
|---|---|---|---|---|
| | Butanol | average | Glucose | average |
| Strain | (mM) | (mM) | (mM) | (mM) |
| KOSIGE-1 | 183.8 | 173.2 | 108.2 | 124.0 |
| KOSIGE-2 | 162.5 | | 139.7 | |
| KOSIGE-1* | 166.9 | 174.0 | 126.0 | 111.7 |
| KOSIGE-2* | 181.0 | | 97.4 | |
| ATCC824 | — | 166.4 | — | 100-130 |
| (WT) | | | | |

### Example 3

### sigG Targeted asRNA

Construction of sigG (CAC1696) Targeted asRNA (pAS-CAC1696)

DNA oligos were designed to target sigG based upon a method previously described (Desai and Papoutsakis, Applied and Environmental Microbiology, 1999. 65(3): p. 936-945). The oligos included 20 base pairs upstream of the start codon (includes the ribosomal binding), the first 13 codons of sigG, a glnA asRNA terminator and 5′-overhangs for directed cloning into a BamHI/KasI double digestion. The oligo sequences are given in the primer and asRNA oligo sequences table and are referred to as CAC1696-asRNA-S and CAC1696-asRNA-AS. The oligos were annealed and ligated into the double digested pSOS95del, and screened for ampicillin resistance in Invitrogen TOP10 E. coli. The resulting asRNA targets the ribosomal binding region and the first 13 codons of the sigG mRNA. It is expressed from the C. acetobutylicum thL promoter, which is a strong promoter.

Morphology Results from sigG Targeted asRNA

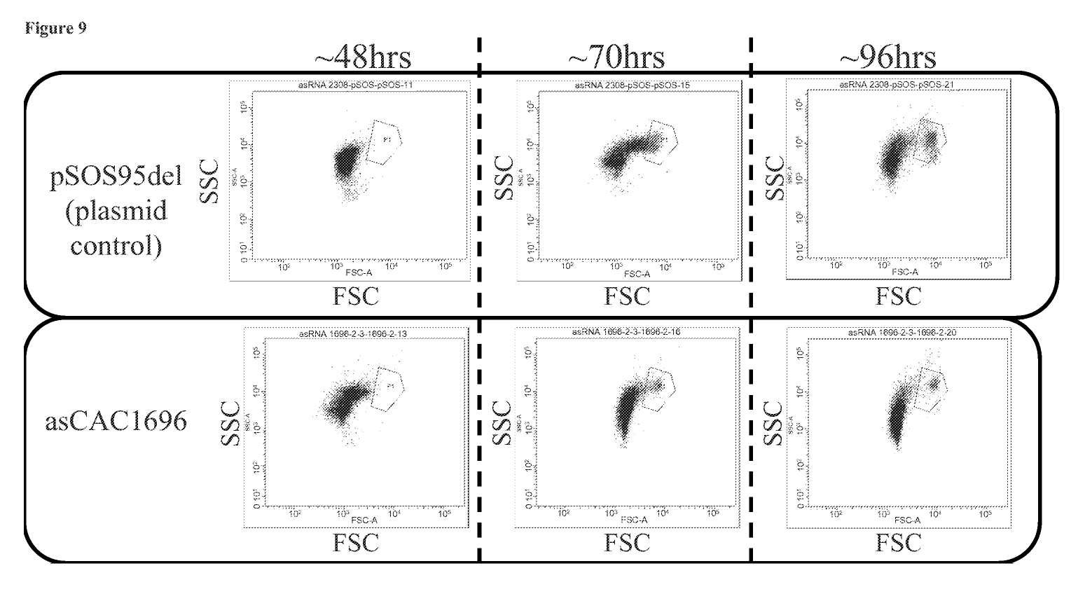

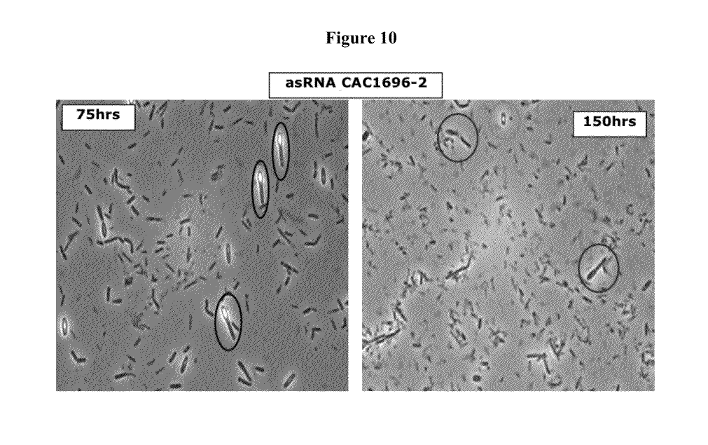

Since asRNA does not completely abolish protein translation, flow cytometry was used to very accurately quantify the percentage of culture that was progressing to late stages of differentiation such as sporulation. In plasmid control cultures an average of 25% of the culture advanced to late stages of sporulation. When two pAS-CAC1696 strains were compared against the pSOS95del plasmid control, >25% of the plasmid control population advanced to late stages of sporulation. The sigG asRNA cultures never rendered more than 6.6% late stage differentiating cells, indicating that the cells in which the asRNA are effective, sporulation was blocked. Refer to Tables 3 & 4 and FIG. 9 for specific results. Additionally, the majority of cells that did advance to late stages of differentiation exhibited abnormal, elongated endospores that are not characteristic of plasmid control or WT cultures. Furthermore, these abnormal endospores appear to abort sporulation at this stage and do not advance to mature spores. This is shown in FIG. 10, where the elongated endospores have obviously lost their phase bright characteristics before completing spore maturation.

HPLC Analysis of sigG asRNA Strains

Significant and reproducible increases in solvent production were witnessed for the sigG asRNA strains compared to the plasmid control. Cultures exhibiting the smallest percentage of differentiating cells generate the greatest amounts of solvent and consume the greatest amount of glucose, refer to Table 3.

TABLE 3
Endpoint HPLC analysis and % differentiation for asCAC1696 versus
pSOS95del cultures. The endpoint HPLC values are from hour 150.
Far less cells advance to late-stages of differentiation for the asRNA
cultures compared to the pSOS95del culture. The as1696-3 exhibited
the smallest percentage of late stage differentiating cells and the
greatest amount of solvent production.
Endpoint HPLC analysis

| | as1696-3 | as1696-2 | pSOS95del |
|---|---|---|---|
| Glucose (mM) | 116.3 | 144.6 | 143.3 |
| Acetone (mM) | 93.4 | 88.7 | 88.7 |
| Butanol (mM) | 157.0 | 142.8 | 140.0 |
| greatest % of late | 5.7 | 6.6 | 25.4 |
| differentiation cells | | | |

TABLE 4
% Late-stage differentiating cells throughout culture life for asCAC1696
versus pSOS95del control cultures. The percentage of late-
differentiation cells remains consistently and significantly lower
for the sigG asRNA cultures.
% late differentiation cells

| Hours after | | | |
|---|---|---|---|
| inoculation | asCAC1696-3 | asCAC1696-2 | pSOS95del |
| 64 | 4.8 | 6.1 | 18.6 |
| 74 | 3.7 | 5.6 | 16 |
| 92.5 | 5.6 | 5.5 | 23.7 |
| 116 | 5.7 | 6.6 | 25.4 |

### Example 4

### sigG Disruption Mutant (KOSIGG)

To target sigG (CAC1696), a 300 bp region of the sigG gene was PCR amplified from C. acetobutylicum genomic DNA with Taq polymerase and the SigG-F/R primer set (SigG-F 5′-GTGGTTATAAACAAGGTTGAAATTTGCGGC-3′; SEQ ID NO:25; SigG-R 5′-CCCTATAATCATCGGAACCGCATAAG-3′; SEQ ID NO:26). The PCR product was cloned into the Invitrogen pCR8-GW-TOPOTA™ cloning plasmid and TOP10 E. coli, resulting in the pCR8-SigG plasmid. pCR8-SigG was linearized by a single ScaI endonuclease digest site in approximately the middle of the sigG gene fragment, resulting in two regions of homology. The first region of homology started at the first nucleotide of the start codon and continued through the second nucleotide of the 49^{th} codon (149 bp total), and the second region of homology continued from the third nucleotide of the 49^{th} codon through the 100^{th} codon (151 bp total). The now linearized pCR8-SigG plasmid was blunt ended, dephosphorylated, ligated to the Th^{r}, and cloned into TOP10 E. coli, resulting in pCR8-SigG/CM/ptB. The pCR8-SigG/CM/ptB was recombined via the Invitrogen Gateway® LR recombination reaction into the pKOREC Destination™ plasmid, via manufacturer suggestions (Invitrogen, Carlsbad, Calif., USA). The final replicating, sigG-targeted plasmid was called pKORSIGG. Disruption of the sigG gene and confirmation of the mutations were carried out as described in Example 2 for sigE disruption.

sigG Disruption Severely Impacts Endospore Architecture and Aborts Sporulation Before Free Spores are Formed

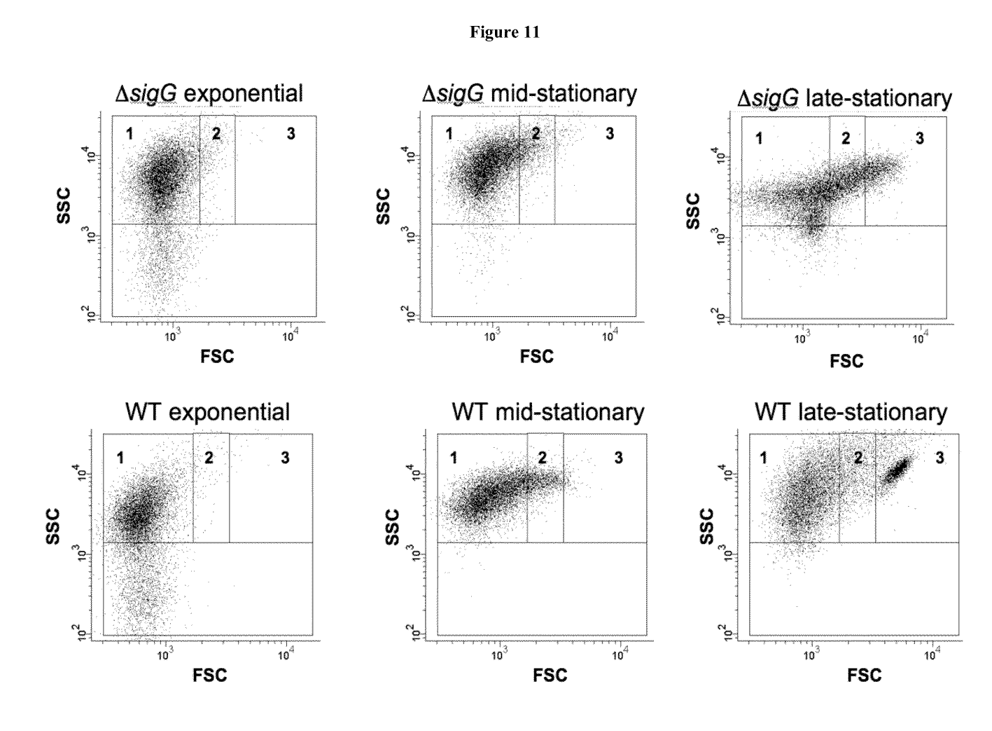

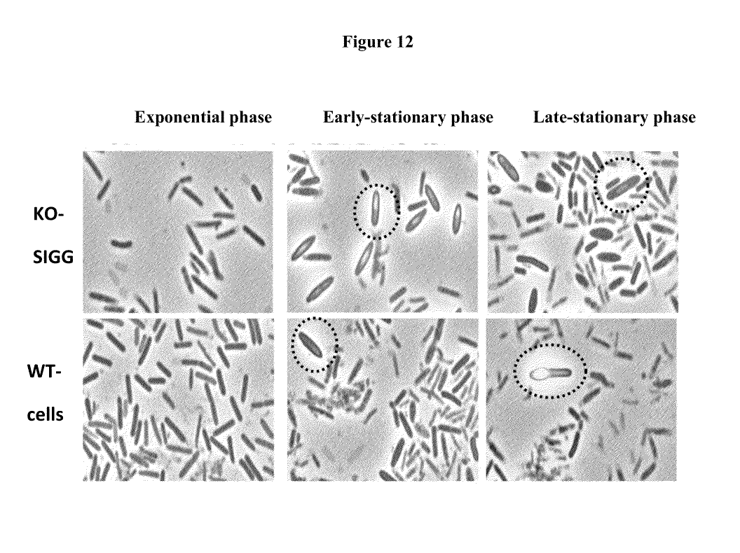

The morphological characteristics of the KOSIGG strain were examined by sporulation assays, FC-LS analysis (see FIG. 11), and phase contrast microscopy (see FIG. 12). Samples for the spore assays were collected at 52, 76 and 120 hours. No colony forming units were obtained for any KOSIGG sample from either spore assay compared to >10,000 CFU/ml for 76 hours or older WT samples. FC-LS analysis of samples taken during exponential, mid- and late-stationary growth phases demonstrated similar LS characteristics as WT cultures through mid-stationary growth phase. In contrast to WT, late-stationary phase KOSIGG cells did not exhibit a condensed, high FSC population (FIG. 11, late stationary) that was separated from all other populations, and which represents free spores. The absence of free spores is consistent with the sporulation-assay results.

Phase contrast microscopy of samples taken between 12-120 hours revealed similar morphology development to that of WT cultures through exponential growth (FIG. 12). However, KOSIGG cultures generated more swollen, phase bright cells similar to clostridial-form cells during early- and mid-stationary phases (FIG. 12). In late-stationary phase, KOSIGG cultures did not exhibit well-defined, phase bright endospores, as observed in WT cultures (FIG. 12). Instead, the clostridial-cell form morphology persisted, and phase dark morphologies with less defined and not as phase-bright endospores were observed (FIG. 12). Phase-bright free spores were never observed in KOSIGG cultures.

Based on the B. subtilis model, σ^{G} expression is exclusively localized to the developing endospore. This was confirmed in C. acetobutylicum using a plasmid control strain (824(pSOS95del)) which sporulates at higher frequencies than the WT strain, thus making observations of σ^{G} expression by intracellular immunofluoresence (ICIF) confocal microscopy more robust. Significant localization of σ^{G} to the endospore in mid-stationary phase 824(pSOS95del) cells was readily observed by confocal ICIF.

sigG Knockout does not Affect Solvent Formation, which is Independent of the Physiological State of the Inoculum

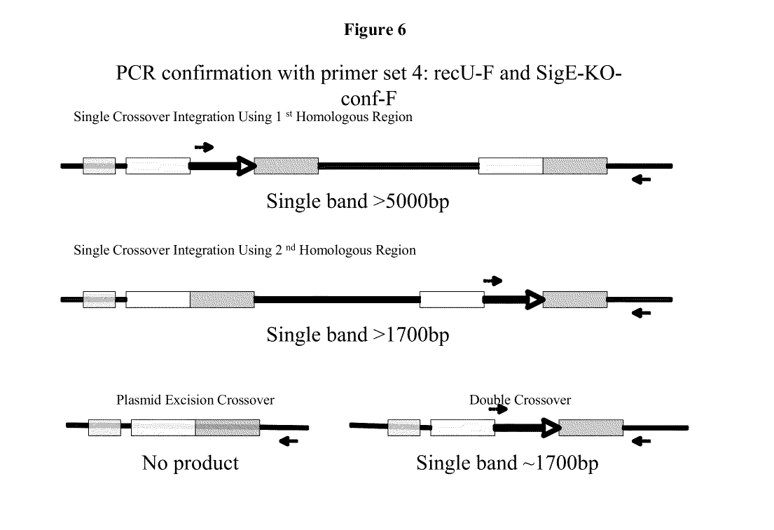

Time course metabolite analyses were performed in biological triplicates for KOSIGG flask cultures and compared to two biological replicates of WT cultures. The KOSIGG colonies were not heat-shocked for preparation of tube culture inocula for the primary flask cultures. The patterns of metabolite production, glucose consumption, and cell growth were similar for KOSIGG and WT cultures (FIGS. 6C & E). However, the KOSIGG cultures re-assimilated acids more completely and produced higher solvent levels. Specifically the KOSIGG cultures produced 179% more ethanol and 29% more butanol than WT cultures, and the KOSIGG cultures always consumed the most glucose. Results indicate that KOSIGG cultures were glucose limited, and by feeding more glucose (fed-batch culture), even greater amounts of solvent can be generated.

All publications, patents, patent applications and accession numbers mentioned in the above specification are herein incorporated by reference in their entirety. Although the invention has been described in connection with specific embodiments, it should be understood that the invention as claimed should not be unduly limited to such specific embodiments. Indeed, various modifications and variations of the described compositions and methods of the invention will be apparent to those of ordinary skill in the art and are intended to be within the scope of the following claims.

1-21. (canceled)

22. A Clostridia cell that does not undergo sporulation, wherein the function of at least one sporulation gene of said bacterial cell is disrupted.

23. The Clostridia cell of claim 22, wherein said Clostridia cell is C. acetobutylicum.

24. The Clostridia cell of claim 22, wherein said sporulation gene is selected from one or more of the group consisting of sigma F, sigma E and sigma G.

25. The Clostridia cell of claim 22, wherein said sporulation gene is knocked out.

26. The Clostridia cell of claim 22, wherein said sporulation gene is mutated.

27. The Clostridia cell of claim 22, wherein expression of said sporulation gene is inhibited.

28. The Clostridia cell of claim 22, wherein expression of said sporulation gene is eliminated.

29. The Clostridia cell of claim 22, wherein expression of said sporulation gene is down-regulated.

30. The Clostridia cell of claim 22, wherein said cell produces solvent.

31. A method for inhibiting sporulation in a Clostridia cell, comprising: contacting said Clostridia cell with a plasmid comprising a nucleic acid that disrupts the function of at least one sporulation gene of said Clostridia cell.

32. The method of claim 31, wherein said nucleic acid knocks out said sporulation gene following homologous recombination.

33. The method of claim 31, wherein said nucleic acid mutates said sporulation gene following homologous recombination.

34. The method of claim 31, wherein said nucleic acid downregulates the expression of said sporulation gene following homologous recombination.

35. The method of claim 31, wherein said nucleic acid integrates into the genome of said bacterium following homologous recombination.

36. The method of claim 31, wherein said Clostridia cell is C. acetobutylicum.

37. The method of claim 31, wherein said sporulation gene is selected from one or more of the group consisting of sigma F, sigma E and sigma G.

38. The method of claim 31, wherein said Clostridia cell produces solvent.
